(19) United States
(12) Patent Application Publication
(10) Pub. No.: US 2022/0395692 A1
(43) Pub. Date: Dec. 15, 2022

(54) In Situ Welding for Feedthrough Pad Attachment

(71) Applicant: BIOTRONIK SE & Co. KG, Berlin (DE)

(72) Inventors: Anthony A. Primavera, Newberg, OR (US); Jutta Muentjes, Erlangen (DE)

(73) Assignee: BIOTRONIK SE & Co. KG, Berlin (DE)

(21) Appl. No.: 17/777,432

(22) PCT Filed: Nov. 17, 2020
(86) PCT No.: PCT/EP2020/082386

§ 371 Date: May 17, 2022

Related U.S. Application Data
(60) Provisional application No. 62/937,296, filed on Nov. 19, 2019.

(30) Foreign Application Priority Data
Jan. 15, 2020 (EP) 20151981.6

(51) Int. Cl. A61N 1/375

(52) U.S. Cl. CPC A61N 1/3754

(57) ABSTRACT

The present invention relates to a method for manufacturing an electrical feedthrough assembly of an electric device, the method comprising the step of: providing an electrical feedthrough assembly comprising an insulating body and at least one electrically connecting element extending through said insulating body, and joining said at least one electrically connecting element with a solderable element, wherein joining is performed by an arc welding process. The invention further relates to a respective feedthrough assembly and an electric device comprising such feedthrough assembly.

### CROSS-REFERENCE TO RELATED APPLICATIONS

This application is the United States National Phase under 35 U.S.C. § 371 of PCT International Patent Application No. PCT/EP2020/082386, filed on Nov. 17, 2020, which claims the benefit of U.S. Provisional Patent Application No. 62/937,296, filed on Nov. 19, 2019 and European Patent Application No. 20151981.6, filed on Jan. 15, 2020, the disclosures of which are hereby incorporated by reference herein in their entireties.

### TECHNICAL FIELD

The present invention relates to a method of manufacture for an electrical feedthrough assembly, a respective feedthrough assembly and an electrical device comprising such feedthrough assembly.

### BACKGROUND

Connections between the inside and outside of implantable electrical devices must maintain hermetic seals to prevent blood and body fluids from entering the device. In addition, the safety of the recipient needs to be maintained to prevent contamination from materials used inside the electrical device. Many applications of implantable electrical devices, such as pulse generators, utilize a ceramic-metallic feedthrough component. The feedthrough allows for electrical signals to be transmitted to and from the electronic module of the implantable electrical device to and from the area of sensing or therapy inside the body, for example, by wires (typically comprised within a lead) connected to the heart in a pacemaker application.

These feedthrough components are often made of a ceramic, which is known as hermetic sealing insulator. In addition, methods exist to seal the ceramic to the conducting wires by melting precious metals such as gold. These processes typically are completed at very high temperatures to ensure the gold melts (1100° C.). Since the signal conducting wires or pins that extend through the feedthrough are potentially exposed to body fluids, the materials needs to be biocompatible. Few known metals are biocompatible and can be sealed to ceramic insulators. On the inside of the device, the conductors need to be connected to the electronic or electrical circuits and power supplies (e.g., battery). Typically, the electrical circuits are connected to circuit boards using tin (Sn) based solders. These allow processing at relatively low temperatures (200° C.) and are inexpensive. Unfortunately, of the known biocompatible feedthrough pin or conductor materials, very few can be joined using Sn based solder. The solder simply does not metallurgically bond with the pin materials at temperatures that the circuits can survive. Typical pin materials are platinum, platinum-iridium and niobium.

In consequence, the dissimilar metals used in circuit board construction and feedthrough pins necessitate the use of contact pads or solderable terminals. These terminals or pads must be attached to the conductor pins of the feedthrough and remain solderable with Sn based solders. The solderable pads are often made of copper or nickel alloys, and are coated to retain solderability.

There are several methods to attach the pad structures to the pins including brazing, mechanical bonding or compression, and laser welding. Each of the aforementioned techniques is typically performed pad by pad.

In the laser welding approach, terminal blocks or pads are brought overtop the pin to ensure that the laser beam can heat both the pin and terminal. Once the parts are in physical proximity, a laser beam pulse is applied to the terminal block. The heat of the subsequent laser energy melts the surface of the terminal or pad and part of the pin. Once heated sufficiently to cause melting of the terminal and part of the pin, the energy is removed and the weld joint is formed upon solidification. However, the laser welding approach has some disadvantages. In general, the approach makes the actual welded connection not inspectable. It is not possible to directly determine if the two materials melted or if only the outer material melted and is touching the pin inside. In addition, the process is highly dependent on the physical touching of the two parts to ensure the melting occurs together.

Furthermore, it is a sequential process, and the equipment is rather expensive. For feedthroughs having multiple rows of pins, the laser beam is obstructed by the outside rows. This limits the process to two rows at most, as the third inside row is shadowed.

In the mechanical compression approach, a terminal block, sleeve or pipe is put over the pin. A series of compressions (swaging) mechanically compresses the outside metal over the inside pin yielding a mechanical compression connection. This approach works well for very large components to be joined, pipes for example, and results in good connections. However, for small diameter and irregular shape wires, the mechanical tooling required to handle the pins is complex. It is extremely difficult to access pins once the feedthrough wires are inserted into the ceramic insulators for fine pitch parts. Thus, this method is limited to pre assembly of pins and pads rather than post assembly processes. In addition, the formed connection is a surface contact or compression connection only and often not metallurgical in nature.

In the brazing approach, a braze material (copper or silver) is used as a joining material between the pin and pad. This layer can be plated or rolled onto the pad surface ahead of time. The parts are then loaded in a fixture that allows physical alignment of the pad to pin so that the pin head or tip touches the pad surface that contains the braze metal. Sufficient heat from a furnace is applied, usually in the range of 1000 to 1200° C., to melt the intermediary material. On cooling, the pad and pin are joined by the braze compound. Typically, very little of the pad or pin actually melts, just the joining compound. One main disadvantage of this approach is that the temperatures needed to connect the dissimilar metals are very high (over 1000° C.). Each cycle of high temperature puts severe stress on the ceramic insulator, and they can develop cracks as a result. In addition, the alignment of the pin to pad is cumbersome and difficult to achieve in production. It is relatively expensive to apply the additional braze coatings to the pads, and the process uses a substantial amount of energy.

The present disclosure is directed toward overcoming one or more of the above-mentioned problems, though not necessarily limited to embodiments that do.

### SUMMARY

Based on this background, it is therefore an objective of the present invention to provide a reliable simple and efficient method for manufacture of feedthrough assemblies, particularly for attaching solderable pads to conductor pins of the feedthrough, which overcomes the above mentioned drawbacks, and furthermore preferably can be automated.

At least this objective is attained by a method having the features of claim 1, a feedthrough assembly having the features of claim 9 and an electric device having the features of claim 13. Appropriate embodiments thereof are stated in the respective dependent claims and the following description.

According to claim 1, a method for manufacturing an electrical feedthrough assembly for an electrical device is provided. The method comprises the steps of:
providing an electrical feedthrough assembly comprising an insulating body and at least one electrically connecting element extending through the insulating body, and
joining the at least one electrically connecting element with a solderable element.

According to the present invention, it is envisioned that joining is performed by arc welding.

Particularly, both the at least one electrically connecting element and the solderable element are made of or comprise an electrically conductive material. Moreover, the solderable element is in particularly joined to one terminus of the at least one electrically connecting element.

The term “arc welding” is used in the context of the present specification within the meaning known to the skilled person. It particularly refers to a joining process, in which an electric arc is generated between two elements to be joined, wherein the electric arc produces heat melting one or both elements, and both elements are pressed together, thereby forming a weld connection.

According to some embodiments of the method of the present invention, joining comprises applying a voltage to at least one electrically connecting element or the solderable element, and approaching the electrically connecting element and the solderable element. Particularly, a voltage source may be connected to the at least connecting element, and a ground electrode to the solderable element, or vice versa. Approaching, particularly in terms of motion speed and/or distance between the connecting element and solderable element particularly, is dependent on the used voltage and the material used for the connecting element and the solderable element.

According to some embodiments of the method of the present invention, the connecting element and/or the solderable element is positioned or aligned before and/or during joining by means of a fixture. Particularly, the fixture is a gantry type fixture. Furthermore, the connecting element or the solderable element may be coupled to the fixture.

The term “gantry type fixture” in the context of the present specification is used in the meaning known to the skilled person. It particularly refers to a fixture that is linearly movable in two or three spatial dimensions.

Accordingly, in some embodiments of the method of the present invention, the connecting element or the solderable element is coupled to a gantry type fixture that is movable in three spatial dimension, i.e. in a X-, Y-, and Z-direction, while the respective other element is coupled to a stationary fixture. In these embodiments, positioning or aligning and approaching of both elements is facilitated by the aforementioned gantry type fixture. Such gantry type fixture may additionally rotatable, i.e. be able to perform a rotation Theta correction.

In some alternative embodiments, the connecting element or the solderable element is coupled to a gantry type fixture that is movable in two spatial dimensions, i.e. in X- and Y-directions, while the respective other element is coupled to a fixture that is movable in one spatial direction, i.e. in Z-direction. In these embodiments, positioning or aligning to two elements to each other is facilitated by the aforementioned gantry type fixture, while approaching of the two elements towards each other is the other fixture.

According to some embodiments of the method of the present invention, the method is automated performed. This may be particularly facilitated by means of the above mentioned gantry type fixture, which may be moved by a robot with high precision. The robot may be particularly configured to move the gantry type fixtures such that at least one connecting element and the solderable element appropriately are approached to each other in order to generate an electric arc (with applied voltage) for arc welding both elements.

According to some embodiments of the method of the present invention, the at least one electrically connecting element is designed as a wire or a pin. In some embodiments, the at least one electrically connecting element comprises or essentially consists of platinum, platinum/iridium, niobium, tungsten, platinum/rhenium or an alloy thereof. In some embodiments, the at least one electrically connecting element is joined, particularly brazed, with the insulating body, particularly with gold a solder. Particularly, the at least one electrically connecting element is hermetically joined with the insulating body. In some embodiments, the electrically connecting element comprises or essentially consists of a platinum/iridium alloy with an iridium content in the range of 5 wt. % to 12 wt. %.

According to some embodiments of the method of the present invention, the solderable element is designed as a terminal block or pad. In some embodiments, the solderable element comprises or essentially consists of nickel, copper or an alloy thereof. In some embodiments, the nickel alloy or the copper alloy may comprises Be and Fe, particularly in small amount, particularly so as to facilitate a rolling or stamping of the solderable element, and/or additionally or alternatively Sn or In, which particularly may act as adhesion promoter between metal layers.

According to some embodiments of the method of the present invention, the insulating body comprises or essentially consists of glass or ceramic.

According to some embodiments of the method of the present invention, the insulating body is surrounded by a metal flange. In some embodiments, the metal flange is brazed to the insulating body, particularly with gold as a solder, and particularly in case of the insulating body is made of glass or ceramic. Particularly, the metal flange is hermetically joined with the insulating body. In some embodiments, the metal flange comprises or essentially consists of titanium or a titanium alloy.

According to some embodiments of the method of the present invention, the feedthrough assembly comprises a plurality of electrically conducting elements extending through the insulation body, wherein the plurality of electrically conducting elements is joined, i.e. arc welded, to a respective plurality of solderable elements in parallel, i.e. in one process step.

According to claim 9, an electrical feedthrough assembly is provided, comprising
an insulating body,
at least one electrically connecting element extending through the insulation body, and,
a solderable element joined to the at least one electrically connecting element.

According to the present invention, it is particularly envisioned that the solderable element is arc welded to the at least one electrically connecting element.

Particularly, the solderable element joined with the at least one electrically connecting element at one of the termini of the electrically connecting element.

According to some embodiments of the electric feedthrough of the present invention, the insulating body is comprises or essentially consists of glass or ceramic.

According to some embodiments, the electrical feedthrough of the present invention further comprises flange surrounding said insulating body. In some embodiments, the flange is made of metal, particularly a biocompatible metal such as titanium or a titanium alloy. In some embodiments, the flange is brazed to the insulating body, particularly with gold as a solder, and particularly in case of the insulating body is made of glass or ceramic. Particularly, the flange is hermetically joined with the insulating body.

According to some embodiments of the electrical feedthrough of the present invention, the electrically connecting element is designed as a wire or a pin. In some embodiments, the electrically connecting element comprises or essentially consists of platinum, platinum/iridium, niobium, tungsten, platinum/rhenium or an alloy thereof. In some embodiments, the at least one electrically connecting element is joined, particularly brazed, with the insulating body, particularly with gold a solder. Particularly, the at least one electrically connecting element is hermetically joined with the insulating body. In some embodiments, the electrically connecting element comprises or essentially consists of a platinum/iridium alloy with an iridium content in the range of 5 wt. % to 12 wt. %.

According to some embodiments of the electrical feedthrough of the present invention, the solderable element is designed as terminal block or pad. In some embodiments, the solderable element comprises or consists of nickel, copper or an alloy thereof. In some embodiments, the nickel alloy or the copper alloy may comprises Be and Fe, particularly in small amount, particularly so as to facilitate a rolling or stamping of the solderable element, and/or additionally or alternatively Sn or In, which particularly may act as adhesion promoter between metal layers.

According to some embodiments, the electrical feedthrough of the present invention comprises a plurality of electrically connecting elements extending through the insulating body, wherein the plurality of electrically connecting elements is joined, i.e. arc welded, to a respective plurality of solderable elements.

According to claim 13, an electric device is provided, wherein the electric device comprises
a housing, and
an electrical feedthrough assembly according to the present invention.

According to some embodiments of the electrical device of the present invention, the housing is hermetically sealed or enclosed.

According to some embodiments of the electrical device of the present invention, the electrical feedthrough assembly is joined, particularly hermetically, to the housing, particularly via a flange of the electrical feedthrough assembly.

According to some embodiments of the electrical device of the present invention, the housing is made of a metal, particularly a biocompatible metal such as, for example, titanium or a titanium alloy. In some embodiments, the flange of the electrical feedthrough assembly is made of a metal, preferable the same metal of the housing, and is welded to the housing. In some embodiments, an insulating body of the electrical feedthrough is made of or comprises glass or ceramic, and is particularly brazed to the flange of the electrical feedthrough assembly, particularly with gold as a solder. Particularly, the flange is hermetically joined with the insulating body.

According to some embodiments, the electrical device further comprises an electronic module or electric component, wherein at least one solderable element of the electrical feedthrough assembly is joined, particularly soldered, to the electronic module or electric component. Non-limiting examples for such electronic module or electric module comprise without being limited to a pulse generator, a circuitry, particularly comprised within a printed circuit board, or an energy storage such as an electrochemical cell, a capacitor, and the like.

According to some embodiments, the electrical device of the present invention is designed as a medical device, particularly an implantable medical device, or a battery or a capacitor. In some embodiments, the electrical device of the present invention is designed as a pacemaker, a cardioverter defibrillator, a loop recorder, or a neuro stimulator.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

Further advantages, features and embodiments of the present invention will be explained hereinafter with reference to the drawings, in which:

FIG. 1 shows a schematic illustration of an electrical feedthrough assembly;

FIG. 2 shows an implantable medical device;

FIG. 3 shows schematic illustration of a general concept of the method of the present invention; and

FIG. 4 shows one embodiment of the method of the present invention.

### DETAILED DESCRIPTION

Ceramic feedthroughs 100 are used to provide a hermetically sealed electrical path from the inside of an implantable electrical device 200 to the outside 203 (body contact side of the device). Typically, discrete wires or conductors 101 are utilized to convey electrical signals and electrical therapy from an electric pulse generator 202 to a lead, electrode or paddle or diagnostic signal the other way round. These wires 101 are often designed to be biocompatible with the blood stream for applications where the device is inside the body. Few conduction materials are body compatible, and almost none of those can be soldered using traditional tin (Sn) based alloys. This necessitates the requirement that a different material, one that is solderable to the internal electrical components 202 be connected to the discrete wires 101 in the feedthrough 100.

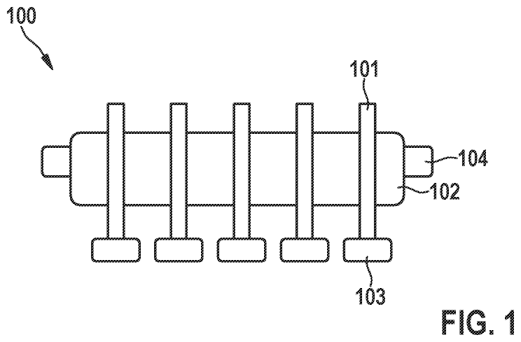

A typical ceramic feedthrough 100 is shown in cross section in FIG. 1. The feedthrough comprises an insulating ceramic body 102, through which a plurality of connecting pins or wires 101 extends. Typically, the pins or wires 101 are made of platinum, platinum/iridium or niobium and are brazed, particularly gold brazed, to the insulating ceramic body 102.

The insulating ceramic body 102 is usually surrounded by and brazed with a metal flange 104, preferable made of titanium or a titanium alloy, for joining the feedthrough 100 with a housing 201 of an electrical device 200 such as an implantable pulse generator. To facilitate soldering the connecting pins or wires 101 to electric or electronic components 202, solderable pads 103 are attached to the connecting pins or wires 101.

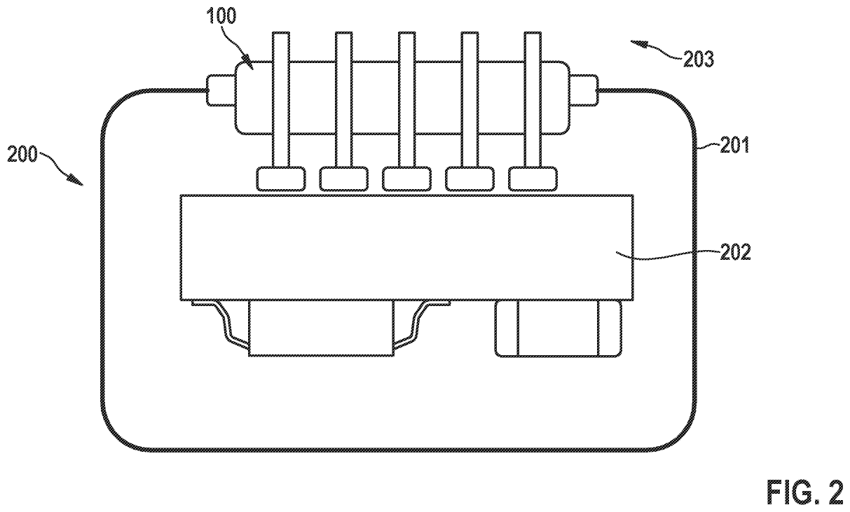

FIG. 2 shows the above mentioned typical use case for an implantable pulse generator 200 utilizing feedthrough technology 100. This implantable pulse generator 200 comprises a housing 201, typically made of titanium or a titanium alloy, wherein the feedthrough 100 is joined to the housing 201 via the flange 104 of the feedthrough 100, preferably by welding.

The implantable pulse generator 200 further comprises electronic and electric components 202 such as power source, integrated circuits, etc. to which the connecting pins 101 are soldered via the attached solderable pads 103.

One objective of the present invention is to establish an automated method of attaching pads 103 to pins 101 in feedthrough components 100, while minimizing the number of high temperature thermal exposures the components experience in fabrication. One goal of this approach is to allow for an assembly process in which the bonding of the pad 103 and pin 101 happens in the automated assembly process itself. The tooling 301, 303 utilized in the feedthrough assembly process can be designed to bring all pins 101 to the same required high voltage potential. The tooling 301, 303 can be brought very near another fixture 302 that contains the solder pads 103. The close proximity and correct current pulses can create an electrical arc. The arc, like a spark plug tip, can reach several thousand degrees, thus melting the surface of each material 101, 103. The arc and simultaneous motion of the feedthrough fixture 301 towards the pad fixture 302 will cause the two features (pad 103 and pin 101) to touch. Once touching, the arc will be quelled since the potential difference is reduced to zero. Once the arc stops, the two parts 101, 103 will solidify and since they are in direct contact, they will be welded together.

Accordingly, the present invention particularly refers to a method for directly attaching solderable pad structures 103 to typical pins 101 utilized in the construction of ceramic type feedthroughs 100. The method provides for a direct, particularly in situ, attachment of the solder pad 103 to the conductor pins 101 during the manufacturing process.

In detail, an electrical circuit is created in the assembly fixture 301, 302 to apply high voltage to each pin 101, particularly by a voltage source 303. The pad 103 is placed in an electrically connected fixture 302 (grounding side of the circuit) such that when the two fixtures 301, 302 are brought in close proximity, an electrical spark or arc is created between the fixture 301 with the feedthrough 100 and the solder pad 103. Once the two fixtures 301, 302 are brought in contact, the arc is quenched and the two surfaces (pin 101 and pad 103) will be connected. The extreme heat of the arc causes the tip of the pin 101 and pad 103 to melt, and once the arc is quenched, the two molten materials 101, 103 solidify together to form a connected structure. One main advantage of this method is that the process can take place at room temperature since the heat is locally applied to each pin/pad via the electrical arc/current. Current methods used for this process typically involve subjecting the entire feedthrough assembly 100 to a high temperature brazing furnace (1100° C.).

The process can be used several ways, however, an electrical test/assembly head is preferred that interfaces with the top or shaft of the pins. This may be a cable or wire assembly that terminates in a connector. The connector may be a block of receptacles (pin connector) that presses overtop of the pins. Particularly, the connector may have cup like shape, in which the pin rests inside the cup wall.

Advantageously, all pads 103 may be attached to the pins 100 simultaneously by contacting all feedthrough pins 100 in parallel. A fixture set 301, 302 may be provided to automate the process, in which a fixture head is movable from assembly to assembly, and with which all feedthrough pins of one assembly may be attached to corresponding pads in parallel.

Alternatively, parallel fixture heads may be provided, with which a plurality of feedthrough assemblies may be manufactured in parallel, particularly facilitating a higher throughput manufacture.

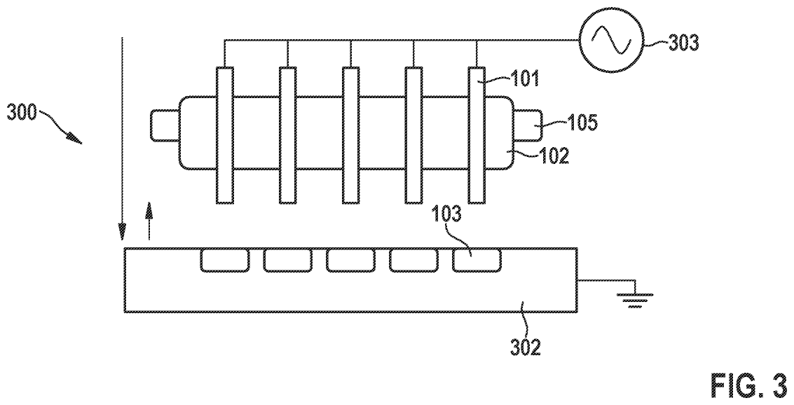

FIG. 3 shows an illustration of the principal approach to connect the pads to the pins. Here, each pin 101 of a feedthrough assembly 100 is connected to a voltage source 303. The solderable pads 103 to be joined are attached or coupled to a fixture 302, by with the solderable pads 103 can be positioned to and aligned with the pins 101. Advantageously, the fixture 302 is made of an electrically conductive material and may serve as a ground electrode. Joining of pins 101 and pads 103 is conducted by applying a current to the pins 101 and moving the pins 101 and the fixtures 302 towards each other as indicated by the arrows. At a certain distance, an arc will emerge between an individual pin 101 and a corresponding pad 103, whereby the tip of the pin and the solderable pad begins to melt. Upon further approaching, the pin 101 and the pads contact each other, whereby the arc is quenched and a metallurgical connection forms.

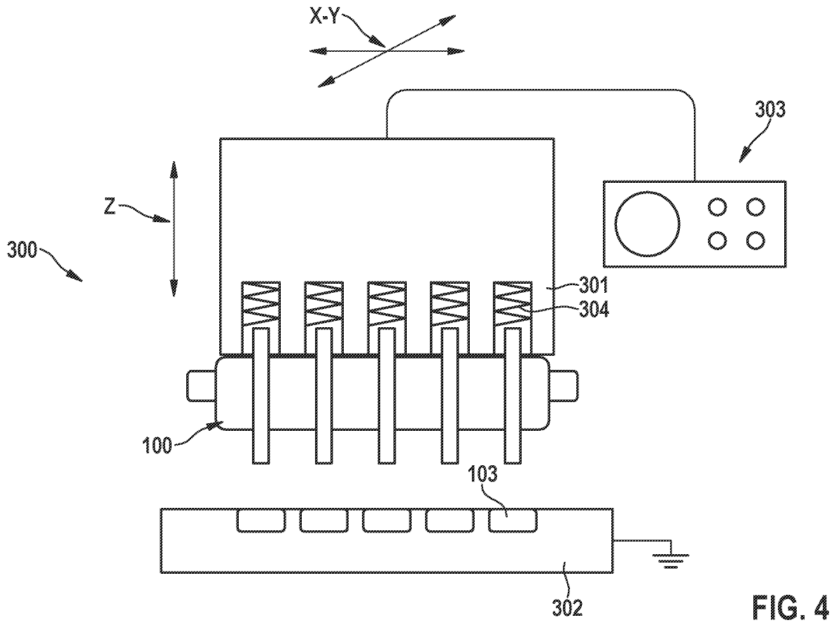

FIG. 4 illustrates one preferred embodiment of the method of the present invention. Therein it is envisioned to couple an overhead X-Y-Z gantry type fixture 301 or machine with an electrical pulse control circuit 303. The overhead gantry type fixture 301 is preferable movable in all three spatial dimensions as indicated by the arrows X and X-Y. Furthermore, the overhead gantry type fixture 303 preferably has connector like features 304, by which the pins 101 on the feedthrough 100 can engage the electrical connections within the fixture 301. Spring contacts 304, for example, may be used to connect the pins 101 with the gantry type fixture 301. A control circuit 303 that is configured to control the electrical voltage and pulse parameters may be used to ramp the potential up in each pin 101 circuit. The time, pulse frequency and amount of current are preferably controllable. The specific parameters for voltage, frequency and current flow depends on the specific material sets being used (pin type, pad type and geometry).

Preferably, an automated test cell or placement robotic cell may be used to pick up the fixtures 303, 302, each feedthrough 100, and control the motion, e.g. in X-Y-X directions, of the process. Advantageously, the electrical high voltage test equipment often used for these types of components may be used to thus combine processes. This automation is particularly useful to reduce manufacturing labor and cost and to provide high volume manufacturing.

The present invention particularly provides the following advantages:

In typical feedthrough manufacturing, the pads 103 are attached to the pins 101 via brazing. The method of the present invention allows for the feedthroughs to be made using a single pass in the high temperature process (gold to ceramic and gold to pin melting). Today, the bulk of the feedthroughs go into the brazing furnace for a second pass to attach the pins 101. Thus reducing labor, cost and stress on the components may be achieved with the method of the present invention. In addition, pad types with different finishes may be attached to the pin without damaging the solderable surface.

The manufacturing process as described above may be combined with voltage testing, e.g. of the pins 101. Advantageously, the same equipment may be used for manufacturing and testing.

The method of the present invention may be automated, particularly as welding occurs in situ in the assembly process.

Simplification of brazing steps is possible with this approach, since at present the brazing is done in two steps as described above. Typically, components are placed into a crucible fixture and sent into the braze furnace to make the basic feed through assembly. A second fixture is used to load the pads on top of the pins and then the parts are put back into the braze furnace a second time. The fixtures and process are complicated due to the many small parts of the fixture. With the method of the present invention, only one brazing step is necessary. Advantageously, due to omitting further brazing steps, thermal stress on the feedthrough assembly may be minimized, particularly since high temperature occurring during the arc welding step are limited to the pin/pad area itself, while the flange/ceramic portion of the assembly may remain at ambient temperature.

The method of the present invention may be applied to multiple types of feedthroughs 100, for example, comprising ceramic as well as glass constructions 102. Thus, the method of the present invention is also applicable for battery and capacity electrode attachment in addition to ceramic feedthroughs for module assembly.

The inspectability of the pin to pad connection is retained by the method of the present invention, as the pin to pad 360 degree fillets are visible.

The method of the present invention is independent of the design of the feedthroughs, unlike laser welding processes, since there is no shadowing or beam path constrictions.

Multiple pins and pads can be joined simultaneously. This process can be massively parallel.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. Method for manufacturing an electrical feedthrough assembly for an electrical device, the method comprising the step of:
providing an electrical feedthrough assembly comprising an insulating body and at least one electrically connecting element extending through said insulating body, and
joining said at least one electrically connecting element with a solderable element,
wherein joining is performed by an arc welding process.

2. The method according to claim 1, wherein joining comprises applying a voltage to said at least one electrically connecting element or said solderable element and approaching said electrically connecting element and said solderable element.

3. The method according to claim 1, wherein said connecting element and/or said solderable element is positioned or aligned before and/or during joining by means of a gantry type fixture, wherein said connecting element and/or said solderable element is coupled to said fixture.

4. The method according to claim 1, wherein said method is automated performed.

5. The method according to claim 1, wherein said at least one electrically connecting element is designed as a wire or a pin, and comprises or essentially consists of platinum, platinum/iridium, niobium, tungsten, platinum/rhenium, or an alloy thereof.

6. The method according to claim 1, wherein said solderable element designed as a terminal block or pad, and comprises or essentially consist of nickel, copper, or an alloy thereof.

7. The method according to claim 1, wherein said insulating body comprises or essentially consists of glass or ceramic.

8. The method according to claim 1, wherein said insulating body is surrounded by a metal flange, which is brazed to said insulating body, wherein said metal flange comprises or essentially consists of titanium or a titanium alloy.

9. Electrical feedthrough assembly, comprising
an insulating body,
at least one electrically connecting element extending through said insulating body, and
a solderable element joined to said at least one electrically connecting element,
wherein said solderable element is arc welded to said at least one electrically connecting element.

10. The electrical feedthrough assembly according to claim 9, further comprising a metal flange, comprising or essentially consisting of titanium or a titanium alloy, surrounding said insulating body, wherein said flange is brazed to said insulating body, and wherein said insulating body is made of or comprises glass or ceramic.

11. The electrical feedthrough assembly according to claim 9, wherein said electrically connecting element is designed as a wire or a pin, and comprises or essentially consists of platinum, platinum/iridium, niobium, tungsten, platinum/rhenium or an alloy thereof.

12. The electrical feedthrough assembly according to claim 8, wherein said solderable element is designed as a terminal block or pad, and comprises or essentially consists of nickel, copper, or an alloy thereof.

13. Electrical device comprising,
a housing, comprising or essentially consisting of titanium or a titanium alloy, and
an electrical feedthrough assembly according to claim 9.

14. The electrical device according to claim 13, further comprising an electronic module or electric component, wherein at least one solderable element of said electrical feedthrough assembly is soldered to said electronic module or electric component.

15. The electrical device according to claim 13, wherein said electric device is designed as a medical device, comprising an implantable medical device, or as a battery, or a capacitor.
